(19) United States
(12) Patent Application Publication
(10) Pub. No.: US 2022/0273605 A1
(43) Pub. Date: Sep. 1, 2022

(54) COMPOSITIONS AND METHODS FOR TREATMENT OF PRESBYOPIA

(71) Applicant: Allergan, Inc., Irvine, CA (US)

(72) Inventors: Michael R. Robinson, Huntington Beach, CA (US); Mohammed Dibas, Corona, CA (US); Anuradha Gore, Aliso Viejo, CA (US)

(21) Appl. No.: 17/597,770

(22) PCT Filed: Jul. 24, 2020
(86) PCT No.: PCT/US20/43534

§ 371 Date: Jan. 21, 2022

Related U.S. Application Data
(60) Provisional application No. 62/879,296, filed on Jul. 26, 2019.

(51) Int. Cl. A61K 31/27; A61K 31/66; A61K 47/02; A61K 47/10; A61K 47/12; A61K 9/00; A61P 27/10

(52) U.S. Cl. CPC A61K 31/27; A61K 31/66; A61K 47/02; A61K 47/10; A61K 47/12; A61K 9/0048; A61P 27/10

(57) ABSTRACT

The present invention is related to topical ophthalmic compositions comprising one or more active components. The active components in the topical ophthalmic compositions include, but are not limited to carbachol, phospholine iodide and pharmaceutically acceptable salts thereof. Also described herein are methods for the treatment of presbyopia, methods for improving near vision in a subject with presbyopia, and methods for reducing pupil diameter in a subject with presbyopia using the topical ophthalmic compositions.

### BACKGROUND OF THE INVENTION

### Field of the Invention

The invention generally relates to methods for treating presbyopia using topical ophthalmic compositions comprising one or more active components including, but not limited to, carbachol, phospholine iodide and pharmaceutically acceptable salts thereof, and a buffer, wherein the topical ophthalmic compositions have a pH of about 3.0 to about 5.5 and generally do not contain a viscosity-enhancing component.

### Background of the Invention

Cholinergic agonists have been used to lower intraocular pressure (“TOP”) so as to treat primary open angle glaucoma. Examples of these medications include pilocarpine, carbachol, phospholine iodide acetylcholine and their respective salt forms. Topical cholinergic agonists act on the ciliary muscle, located in the ciliary body of the eye (Levin et al., Adler's Physiology of the Eye, 11th edition by Saunders Elsevier (Edinburgh), pp. 56, 57, and 509-510), thereby causing it to contract, which in turn opens the trabecular meshwork (Id, pp. 44, 45, and 289-291). This can facilitate the rate at which aqueous humor leaves the eye and the net result is a reduction of the intraocular pressure (“TOP”) in patients with primary open angle glaucoma. Muscarinic mediated ciliary muscle contraction also lead to relaxation of zonule fibers leading the thickening of lens to focus near objects on retina (accommodation). These agents also act on the muscarinic cholinergic receptors found on the iris sphincter muscle, causing the muscle to contract, resulting in pupil constriction (i.e., miosis) (Levin et al., Adler's Physiology of the Eye, 11th edition by Saunders Elsevier (Edinburgh), pp. 56, 57, and 509-510).

In patients approximately 40 years old or greater, there is a gradual loss in the ability to focus (particularly at close distance) primarily due to stiffening of the lens in the eye, a refractive condition known as presbyopia (Levin et al., Adler's Physiology of the Eye E-Book, 11^{th} edition by Saunders Elsevier (Edinburgh), pp. 59-61). Application of cholinergic agonists in these patients is beneficial as the miosis resulting from sphincter muscle contraction creates a “pin-hole effect” that may potentially improve the near and intermediate vision by increasing the depth of field. These cholinergic agonists can thus be used for the treatment of presbyopia, although most effective dosing frequency and dose concentrations have not been defined. The present disclosure addresses this need. Furthermore, currently available commercial ophthalmic formulations are typically formulated with viscosity enhancing polymers (Ritch et al., The Glaucomas, Mosby (St. Louis), p. 517, 1989). Viscosity enhancing polymers are used to increase the corneal residency time of the active ingredients of the ophthalmic compositions to increase penetration into the eye, since the active ingredients are diluted with tears and nasolacrimal duct drainage. However, the viscosity due to added polymers in commercially available ophthalmic formulations often results in adverse effects such as vision blur that limit the use of such ophthalmic formulations (Hall et al., Optom. Vis. Sci., 88, pp. 872-880, 2011). Additionally, in some cases, the added polymers cause ocular discomfort such as eye pain, brow ache, blurry vision, light sensitivity, ocular stinging, and ocular itching. These adverse effects result in decreased patient compliance. Thus, there is a need in the art for improved topical ophthalmic compositions that optimize ocular comfort and improve patient compliance by reducing or eliminating the adverse effects commonly associated with currently available commercial ophthalmic formulations, while not compromising therapeutic potency. The present disclosure additionally addresses this need.

### SUMMARY OF THE INVENTION

Certain embodiments disclosed herein relate to topical ophthalmic compositions comprising one or more active components. The active components in the topical ophthalmic compositions may include, but are not limited to carbachol, phospholine iodide, and pharmaceutically acceptable salts thereof. Moreover, the topical ophthalmic compositions preferably include a buffer, have a pH of about 3.0 to about 5.5 and do not contain a viscosity-enhancing component.

Certain embodiments disclosed herein further provide topical ophthalmic compositions comprising one or more active components. The active components in the topical ophthalmic compositions may include, but are not limited to carbachol, phospholine iodide, and pharmaceutically acceptable salts thereof. Moreover, the topical ophthalmic preferably compositions include a buffer, have a pH of about 3.0 to about 5.5 and a viscosity from about 1 centipoise (cps) to about 10 cps.

Certain embodiments disclosed herein also provide methods of treating presbyopia in a subject in need of treatment thereof, comprising administering to at least one eye of the subject a therapeutically effective amount of one or more topical ophthalmic compositions comprising one or more active components. The active components in the topical ophthalmic compositions may include, but are not limited to carbachol, phospholine iodide, and pharmaceutically acceptable salts thereof. The topical ophthalmic compositions preferably also include a buffer.

Certain embodiments disclosed herein further provide methods of improvement of near vision in a subject with presbyopia in need thereof. The methods comprise administering to at least one eye of the subject a therapeutically effective amount of one or more topical ophthalmic compositions comprising one or more active components. The active components in the topical ophthalmic compositions may include, but are not limited to carbachol, phospholine iodide, and pharmaceutically acceptable salts thereof. The topical ophthalmic compositions preferably also include a buffer.

Certain embodiments disclosed herein additionally provide methods for reducing pupil diameter in a subject with presbyopia in need thereof. The methods comprise administering to at least one eye of the subject a therapeutically effective amount of one or more topical ophthalmic compositions comprising one or more active components. The active components in the topical ophthalmic compositions may include, but are not limited to carbachol, phospholine iodide, and pharmaceutically acceptable salts thereof. The topical ophthalmic compositions preferably also include a buffer.

### BRIEF DESCRIPTION OF THE FIGURES

FIG. 1 shows the effect of Pilocarpine on pupil diameter in rabbit animal model.

FIG. 2 shows the effect of carbachol on pupil diameter in rabbit animal model.

### DETAILED DESCRIPTION OF THE INVENTION

Unless defined otherwise, all technical and scientific terms used herein have the same meaning as commonly understood by one of ordinary skill in the art to which the subject matter pertains.

As used in this specification and the appended claims, the singular forms “a,” “an” and “the” include plural referents unless the context clearly dictates otherwise.

The invention provides topical ophthalmic compositions comprising one or more active components. The term “topical” as used herein refers to a composition intended for direct application to the corneal surface of an eye of a subject in need thereof. Such application may be accomplished, for example, via an eyedrop dispenser. The term “topical” does not include injections to the eye of a subject (e.g., anterior chamber injections).

The term “ophthalmic composition” or “ophthalmic compositions of the invention” as used herein refers to compositions suitable for application to an eye of a subject, which are in such form as to permit the biological activity of the one or more active components (e.g., carbachol, phospholine iodide) to be effective, and which contain no additional components that are unacceptably toxic to the subject to which the composition would be administered. Such ophthalmic compositions will generally be sterile. Thus, for topical application to the eye, the ophthalmic compositions of the present invention will generally be formulated as sterile aqueous compositions (e.g., suspensions, solutions, emulsions or the like) and typically include at least 70 w/v %, more typically 80 w/v % and even more typically at least 90 or 95 w/v % purified water. Such ophthalmic compositions may be in the form of liquid preparations, e.g., eye drops. The ophthalmic compositions may be suitable for single-dose or multiple-dose topical application. The ophthalmic compositions suitable for multi-dose topical application are often disposed in a dispenser (e.g., an eye dropper), which can dispense the ophthalmic composition (e.g., as individual drops) to the corneal surface of the eye.

As used herein, the term “active component” refers to a component of the topical ophthalmic compositions of the invention which is responsible for the therapeutic effect of the composition, whereas the other components of the composition (e.g., excipients, carriers, and diluents) are not responsible for the therapeutic effect of the composition, even if they have other functions in the composition which are necessary or desired as part of the formulation (such as lubrication, pH control, emulsification, stabilization, preservation, and other functions). In some embodiments, the active components have therapeutic activity for the treatment of an ocular condition, such as presbyopia, or for improving near vision in a subject with presbyopia, or for reducing pupil diameter in a subject with presbyopia.

The active components in the topical ophthalmic compositions of the invention include, but are not limited to, carbachol and phospholine iodide. Carbachol is a cholinergic parasympathomimetic drug, and is sometimes referred to as carbamoylcholine chloride or carbamylcholine chloride, and is commonly represented with the following structure:

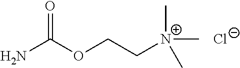

Carbachol typically presents as a positively-charged quaternary ammonium compound with a chloride counterion as depicted above, though other salt forms are possible.

Phospholine iodide is an irreversible acetylcholinesterase inhibitor, and is sometimes referred to as echothiophate or (2-mercaptoethyl) trimethylammonium iodide O,O-diethyl phosphorothioate. It has the following structure:

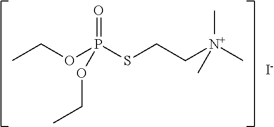

Phospholine is a quaternary ammonium salt, and typically presents with an iodide counterion as depicted above, though other salt forms are possible.

In certain embodiments, at least one of the one or more active components in the compositions of the present invention is/are present at a concentration of at least about 0.01% w/v. In other embodiments, at least one of the one or more active components is present at a concentration of less than about 0.01% w/v. In additional embodiments, the one or more active components are each present at a concentration of at least about 0.01% w/v. In certain aspects, at least one of the one or more active components is present at a concentration from about 0.01% w/v to about 20% w/v. In other aspects, the one or more active components are each present at a concentration from about 0.01% w/v to about 20% w/v. In some embodiments, at least one of the one or more active components is present at a concentration from about 0.01% w/v to about 10% w/v. In other embodiments, the one or more active components are each present at a concentration from about 0.01% w/v to about 10% w/v. In certain embodiments, at least one of the one or more active components is present at a concentration from about 0.03% w/v to at least about 3% w/v. In other embodiments, the one or more active components are each present at a concentration from about 0.03% w/v to at least about 3% w/v. In additional embodiments, at least one of the one or more active components is present at a concentration from about 0.1% w/v to at least about 1% w/v. In further embodiments, the one or more active components are each present at a concentration from about 0.1% w/v to at least about 1% w/v.

In certain embodiments, the topical ophthalmic compositions of the invention comprise carbachol as an active component. In certain aspects, carbachol is the sole active component present in the topical ophthalmic compositions of the invention. In some embodiments, carbachol is present as a pharmaceutically acceptable salt. In some embodiments, when carbachol is part of a topical ophthalmic composition, the compound is the sole active component which has therapeutic activity for the treatment of ocular conditions or improvement of vision parameters. For example, this might include the treatment of an ocular condition including, but not limited to, presbyopia, or for improving near vision in a subject with presbyopia, or for reducing pupil diameter in a subject with presbyopia. In certain aspects, the topical ophthalmic compositions comprise at least about 0.01% (w/v) carbachol. In other aspects, the topical ophthalmic compositions comprise less than about 0.01% (w/v) carbachol. In some embodiments, the topical ophthalmic compositions comprise carbachol at a concentration from about 0.01% (w/v) to about 20% (w/v). In other embodiments, the topical ophthalmic compositions comprise carbachol at a concentration from about 0.01% (w/v) to about 10% (w/v). In yet other embodiments, the topical ophthalmic compositions comprise carbachol at a concentration from about 0.03% (w/v) to about 3% (w/v). In additional embodiments, the topical ophthalmic compositions comprise carbachol at a concentration from about 0.1% (w/v) to about 1% (w/v). In specific embodiments, the topical ophthalmic compositions comprise carbachol at a concentration of about 0.6% (w/v). Other amounts of carbachol that may be used include 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.10% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), 0.20% (w/v), 0.25% (w/v), 0.30% (w/v), 0.40% (w/v), 0.45% (w/v), 0.50% (w/v), 0.55% (w/v), 0.60% (w/v), 0.65% (w/v), 0.70% (w/v), 0.75% (w/v), 0.80% (w/v), 0.90% (w/v), 0.95% (w/v), 1.0% (w/v), 1.1% (w/v), 1.2% (w/v), 1.25% (w/v), 1.50% (w/v), 1.75% (w/v), 2.0% (w/v), 2.25% (w/v), 2.5% (w/v), 3.0% (w/v), 3.25% (w/v), 3.5% (w/v), 3.75% (w/v), 4% (w/v), 4.5% (w/v), 5% (w/v), and ranges and amounts between any of these selected amounts of carbachol.

In other embodiments, the topical ophthalmic compositions of the invention comprise phospholine iodide as an active component. In certain aspects, phospholine iodide is the sole active component present in the topical ophthalmic compositions of the invention. In some embodiments, phospholine iodide is present as a pharmaceutically acceptable salt. In some embodiments, when phospholine iodide is part of a topical ophthalmic composition, the compound is the sole active component which has therapeutic activity for the treatment of ocular conditions or improvement of vision parameters. For example, this might include the treatment of an ocular condition or for improving a vision parameter. In certain aspects, the topical ophthalmic compositions comprise at least about 0.01% (w/v) phospholine iodide. In other aspects, the topical ophthalmic compositions comprise less than about 0.01% (w/v) phospholine iodide. In some embodiments, the topical ophthalmic compositions comprise phospholine iodide at a concentration from about 0.01% (w/v) to about 20% (w/v). In other embodiments, the topical ophthalmic compositions comprise phospholine iodide at a concentration from about 0.01% w/v to about 10% w/v. In additional embodiments, the topical ophthalmic compositions comprise phospholine iodide at a concentration from about 0.01% (w/v) to about 0.25% (w/v). In specific embodiments, the topical ophthalmic compositions comprise phospholine iodide at a concentration of about 0.06% (w/v). Other amounts of phospholine iodide that may be used include 0.001% (w/v), 0.025% (w/v), 0.005% (w/v), 0.075% (w/v), 0.01% (w/v), 0.02% (w/v), 0.03% (w/v), 0.04% (w/v), 0.05% (w/v), 0.055% (w/v), 0.06% (w/v), 0.065% (w/v), 0.07% (w/v), 0.08% (w/v), 0.09% (w/v), 0.10% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), 0.20% (w/v), 0.25% (w/v), 0.30% (w/v), 0.40% (w/v), 0.45% (w/v), 0.50% (w/v), 0.55% (w/v), 0.60% (w/v), 0.65% (w/v), 0.70% (w/v), 0.75% (w/v), 0.80% (w/v), 0.90% (w/v), 0.95% (w/v), 1.0% (w/v), 1.1% (w/v), and ranges and amounts between any of these selected amounts of phospholine iodide.

The topical ophthalmic compositions may also include pharmaceutically acceptable salts of the active components. As used herein, the term “pharmaceutically acceptable salts” refers to salts of the one or more active agents of the topical ophthalmic compositions of the invention that are substantially non-toxic to living organisms, e.g., subjects in need of the topical ophthalmic compositions. Typical pharmaceutically acceptable salts include those salts prepared by reaction of the one or more active components of the invention with an inorganic or organic acid, or an organic base, depending on the substituents present on the one or more active components of the invention.

Inorganic acids which may be used to prepare pharmaceutically acceptable salts of the active components include, but are not limited to, hydrochloric acid, phosphoric acid, sulfuric acid, hydrobromic acid, hydroiodic acid, phosphorous acid and the like. Organic acids which may be used to prepare pharmaceutically acceptable salts include, without limitation, aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include, but are not limited to, hydrochloride, hydrobromide, nitrate, sulfate, pyrosulfate, bisulfate, sulfite, bisulfite, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, and maleate. Suitable pharmaceutically acceptable salts may also be formed by reacting the active components with an organic base such as methylamine, ethylamine, ethanolamine, lysine, ornithine and the like. Pharmaceutically acceptable salts include the salts formed between carboxylate or sulfonate groups that may be found on some of the active components and inorganic cations, such as sodium, potassium, ammonium, or calcium, or such organic cations as isopropylammonium, trimethylammonium, tetramethylammonium, and imidazolium. All of these salts may be prepared by conventional means from the active components of the invention by reacting, for example, the appropriate acid or base with the active components of the invention.

The topical ophthalmic compositions of the invention also include a suitable buffer. As used herein, the term “buffer” refers to a component of a solution that resists changes in pH of the solution when an acid or alkali is added to it. Buffers typically involve a weak acid or alkali together with one of its salts. For example, a buffer may comprise one or more of sodium phosphate dibasic heptahydrate, sodium phosphate monobasic monohydrate, sodium hydroxide, or hydrochloric acid. In certain embodiments, the buffer comprises monobasic and dibasic sodium phosphate. The quality of a buffer is determined by its buffer capacity, i.e. its resistance to changes in pH when strong acids or bases are added. In other words, the buffer capacity corresponds to the amount of H⁺ or OH⁻ ions that can be neutralized by the buffer. Buffer capacity is related to the buffer concentration. A graph described by the relation of the pH to the addition of H⁺/OH⁻ ions is called the titration curve. The point of inflection of the curve corresponds to the pKa value of the buffer. The buffer capacity of a buffer is at its maximum at the pKa value. The pKa value of a buffer therefore corresponds to the mid-point of the pH range covered by the buffer and represents the point at which the concentration of acid and base is the same. In the area of this pH range, therefore, relatively large amounts of H⁺/OH⁻ ions result in only small changes in pH. Therefore, a buffer with more than one pKa resists changes to the pH of a solution over a broad range of H⁺/OH⁻ ions. Examples of buffers with more than one pKa include, but are not limited to, citrate buffer and phosphate buffer.

A buffer suitable for use in the topical ophthalmic compositions of the invention is one that stabilizes the stored compositions by maintaining the compositions at a low pH (e.g., pH of about 3.0 to about 5.5), but quickly equilibrates to physiological pH (i.e., pH of about 7.0) when the compositions are administered to the surface of an eye. Examples of suitable buffers include, but are not limited to, sodium citrate dehydrate buffer, phosphate buffer, borate buffer, borate citrate buffer, lactate buffer and citrate buffer.

In certain embodiments, the buffer is present at a concentration of less than about 0.001% (w/v). In other embodiments, the buffer is present at a concentration of at least about 0.001% (w/v). In other embodiments, the buffer is present at a concentration from about 0.001% (w/v) to about 1% (w/v). In specific embodiments, the buffer is a sodium citrate dihydrate buffer. In certain aspects, the sodium citrate dihydrate buffer is present at a concentration from about 0.01% (w/v) to about 0.1% (w/v). In specific aspects, the sodium citrate dihydrate buffer is present at a concentration of about 0.015% (w/v).

A buffer may control the pH of the topical ophthalmic compositions of the invention. In certain embodiments, the topical ophthalmic compositions of the invention have a pH of lower than about 7.4. In other embodiments, the topical ophthalmic compositions of the invention have a pH of lower than about 7.0, lower than about 6.5, lower than about 6.0, lower than about 5.5, lower than about 5.0, lower than about 4.5, lower than about 4.0, lower than about 3.5 lower than about 3.0, lower than about 2.5, lower than about 2.0, lower than about 1.5, or lower than about 1.0. In certain aspects, the pH of topical ophthalmic compositions of the invention is in the range of about 1.0 to about 6.5, about 1.0 to about 6.0, about 1.0 to about 5.5, about 1.5 to about 5.5, about 2.0 to about 5.5, about 2.5 to about 5.5, about 3.0 to about 5.5, about 3.5 to about 5.5, about 4.0 to about 5.5, about 4.5 to about 5.5, or about 5.0 to about 5.5. In certain embodiments, the pH of topical ophthalmic compositions of the invention is in the range of about 3.0 to about 5.5. In specific embodiments, the pH of topical ophthalmic compositions of the invention is 5.0. The pH of the ocular topical ophthalmic compositions of the invention unexpectedly reduces or eliminates ocular discomfort commonly associated with commercially available topical ophthalmic compositions. The ocular discomfort symptoms may include, without limitation, eye pain, brow ache, blurry vision, light sensitivity, ocular stinging, and ocular itching.

The topical ophthalmic compositions of the invention may or may not contain a secondary buffering agent. In certain aspects, the secondary buffering agent includes, without limitation, citrate buffer and acetate buffer. In certain embodiments, the secondary buffering agent is present at a concentration of at least about concentration of less than about 0.001 mM. In other embodiments, the secondary buffering agent is present at a concentration of at least about 0.001 mM. In some embodiments, the secondary buffering agent is present at a concentration from about 0.01 mM to 1M. In specific embodiments, the secondary buffering agent is present at a concentration from about 1 mM to about 100 mM.

Viscosity enhancing agents are used in the majority of topical ophthalmic compositions to increase the corneal residency time of the active ingredients of the ophthalmic compositions to increase penetration into the eye since the active ingredients are diluted with tears and nasolacrimal duct drainage. However, viscosity enhancing agents have the side effects of blurry vision and, in some cases, irritation. Thus, contrary to the currently available commercial topical ophthalmic compositions, in select specific embodiments, some of the topical ophthalmic compositions of the invention do not contain viscosity enhancing components. Unexpectedly, the topical ophthalmic compositions of the invention demonstrated excellent efficacy results without the use of viscosity enhancing components. As used herein, the term “viscosity” of a topical ophthalmic composition of the invention is used as it normally is used for liquids and means a measure of the liquid's resistance to deformation at a given rate. Thus, viscosity is a quantity expressing the magnitude of internal friction, as measured by the force per unit area resisting a flow in which parallel layers of the topical ophthalmic compositions, unit distance apart, have unit speed relative to one another. A fluid that has no resistance to shear stress is known as an ideal or inviscid fluid. Zero viscosity is observed only at very low temperatures in superfluids. Otherwise, the second law of thermodynamics requires all fluids to have positive viscosity; such fluids are technically said to be viscous or viscid. A fluid with a relatively high viscosity, such as pitch, may appear to be a solid. In certain embodiments, the topical ophthalmic compositions of the invention have a viscosity from about 1 centipoise (cps) to about 10 cps. In certain aspects, the topical ophthalmic compositions of the invention have a viscosity close to that of pure water (1 cps). In specific aspects, the topical ophthalmic compositions of the invention have a viscosity of about 1 cps.

As used herein, the term “viscosity enhancing component” refers to any substance that increases the viscosity of the topical ophthalmic compositions of the invention. A viscosity enhancing component may be a polymer including, but not limited to hypromellose, carboxymethyl cellulose, hydroxyethyl cellulose, hydroxymethyl cellulose, methylcellulose, methyl cellulose 4000, hydroxypropyl cellulose, hydroxypropylmethyl cellulose, hydroxyl propyl methyl cellulose 2906, carboxypropylmethyl cellulose, hydroxypropylethyl cellulose, and hydroxyethyl cellulose, polyethylene glycol, polyvinyl alcohol, pyrrolidone, polyvinyl pyrrolidone, gellan, carrageenan, alginic acid, carboxyvinyl polymer, glycerol, acrylic polymers (e.g., carbomer, polycarbophil), hyaluronic acid, hydroxypropyl-guar (hp-guar), xanthan gum, alginate, chitosan, gelrite, dextran, or combinations thereof. In specific embodiments, the compositions of the present invention do not contain any of the above-listed polymeric viscosity enhancing agents, even if the compound(s) could be used to serve another purpose other than viscosity enhancement. In other specific embodiments, the compositions of the present invention contain only trace amounts of any of the above-listed polymeric viscosity enhancing agents, even if the compound(s) could be used to serve another purpose other than viscosity enhancement. As used herein, a trace amount means a concentration of about 100 ppm (100 micrograms per gram to 100 micrograms per milliliter) or less. In other specific embodiments, the compositions of the present invention may contain small amounts of any of the above-listed polymeric viscosity enhancing agents, even if the compound(s) could be used to serve another purpose other than viscosity enhancement, provided that the viscosity of the resulting composition is about 20 cps or less, about 15 cps or less, about 10 cps or less, about 5 cps or less or about 2 cps or less. In other aspects, a viscosity enhancing component may be non-polymeric. In specific embodiments, the compositions of the present invention do not contain any of the above-listed non-polymeric viscosity enhancing agents, even if the compound(s) could be used to serve another purpose other than viscosity enhancement. In other specific embodiments, the compositions of the present invention contain only trace amounts of any of the above-listed non-polymeric viscosity enhancing agents, even if the compound(s) could be used to serve another purpose other than viscosity enhancement. In other specific embodiments, the compositions of the present invention may contain small amounts of any of the above-listed non-polymeric viscosity enhancing agents, even if the compound(s) could be used to serve another purpose other than viscosity enhancement, provided that the viscosity of the resulting composition is about 20 cps or less, about 15 cps or less, about 10 cps or less, about 5 cps or less or about 2 cps or less.

The topical ophthalmic compositions of the invention may further include one or more osmolality agents in an amount that renders the topical ophthalmic compositions of the invention roughly isotonic. “Osmolality” is a measure of the total number of dissolved particles in a given volume of a solution. As used here, the term “osmolality agent” include any compound or substance useful for adjusting the osmolality of a topical ophthalmic composition. Examples of osmolality agents include, but are not limited to, salts, particularly sodium chloride or potassium chloride, organic compounds such as propylene glycol, mannitol, sorbitol, dextrose, and glycerin. In certain embodiments, the osmolality agents of the topical ophthalmic compositions of the invention include, but are not limited to, glycerin, propylene glycol, mannitol, sorbitol, sodium chloride, potassium chloride and dextrose.

“Tonicity” is a measure of the effective osmotic pressure gradient, as defined by the water potential of two solutions separated by a semipermeable membrane. In other words, tonicity is the relative concentration of solutes dissolved in solution which determine the direction and extent of diffusion. The term is commonly used when describing the response of cells immersed in an external solution. Unlike osmotic pressure, tonicity is influenced only by solutes that cannot cross the membrane, as only these exert an effective osmotic pressure. Solutes able to freely cross the membrane do not affect tonicity because they will always be in equal concentrations on both sides of the membrane. There are three classifications of tonicity that one solution can have relative to another: hypertonic, hypotonic, and isotonic. A solution is “isotonic” when its effective osmole concentration is the same as that of another solution. In biology, the solutions on either side of a cell membrane, for example, are isotonic if the concentration of solutes outside the cell is equal to the concentration of solutes inside the cell.

In certain embodiments, the one or more osmolality agents is selected from the group consisting of glycerin, propylene glycol, mannitol, sorbitol, sodium chloride, potassium chloride, and dextrose. The amount of an osmolality agent may vary depending upon whether the topical ophthalmic compositions are isotonic, hypertonic, or hypotonic. In certain embodiments, the amount of an osmolality agent such as those listed above may be at least about 0.0001% (w/v) up to about 1% (w/v), about 2% (w/v), about 5% (w/v), about 10% (w/v), or about 20% (w/v). In some embodiments, at least one of the one or more osmolality agents is present at a concentration of at least about 0.0001% (w/v). In other embodiments, the one or more osmolality agents are each present at a concentration of at least about 0.0001% (w/v). In some embodiments, at least one of the one or more osmolality agents is present at a concentration from about 0.001% (w/v) to about 20% (w/v). In other embodiments, the one or more osmolality agents are each present at a concentration from about 0.001% (w/v) to about 20% (w/v). In additional embodiments, at least one of the one or more osmolality agents is present at a concentration from about 0.001% (w/v) to about 5% (w/v). In further embodiments, the one or more osmolality agents are each present at a concentration from about 0.001% (w/v) to about 5% (w/v). In yet other embodiments, at least one of the one or more osmolality agents is present at a concentration from about 0.001% (w/v) to about 2.5% (w/v). In additional embodiments, the one or more osmolality agents are each present at a concentration from about 0.001% (w/v) to about 2.5% (w/v). In still other embodiments, at least one of the one or more osmolality agents is present at a concentration from about 0.001% w/v to about 1% w/v. In further embodiments, the one or more osmolality agents are each present at a concentration from about 0.001% (w/v) to about 1% (w/v). In certain embodiments, the osmolality agent is sodium chloride. In certain aspects, the sodium chloride is present at a concentration from about 0.1% (w/v) to about 0.9% (w/v). In specific embodiments, the sodium chloride is present at a concentration of about 0.37% (w/v).

The topical ophthalmic compositions of the invention may further include a strong acid or a strong base. Examples of strong acids and strong bases are well known in the art and include, without limitation, NaOH, KOH, HCl, and H₂SO₄. In specific aspects, the topical ophthalmic compositions of the invention further comprise NaOH or HCl.

The topical ophthalmic compositions of the invention may also include boric acid. In certain embodiments, the boric acid is present at a concentration from about 0.01% (w/v) to about 5% (w/v). In additional embodiments, the boric acid is present at a concentration from about 0.1% (w/v) to about 1.5% (w/v). In specific embodiments, the boric acid is present at a concentration of about 1% (w/v).

The topical ophthalmic compositions of the invention may be packaged for single use, and contain no preservative or essentially no preservative. Alternatively, the topical ophthalmic compositions of the invention may be packaged for multiple uses, and comprise a suitable preservative to prevent contamination over multiple uses. As used herein, the term “preservative” means any substance that prevents or retards contamination in the form of bacterial or fungal growth in the topical ophthalmic solutions of the invention. Examples of suitable preservatives include, but are limited to, benzalkonium chloride (BAK), Polyquaternium-1 (Polyquad®), chlorobutanol, and a stabilized oxychloro complex comprising chlorite, chlorate and chlorine dioxide (also known as stabilized chlorine dioxide). Stabilized oxychloro complex, also known as Purite®, may be described as an aqueous solution of sodium chlorite (NaClO₂). U.S. Pat. No. 5,424,078, which is incorporated herein by reference in its entirety, further discusses the use of stabilized oxychloro complex as a preservative for topical ophthalmic compositions.

In certain embodiments, the preservative is present at a concentration of at least about 1 ppm. In other embodiments, the preservative is present at a concentration of less than about 1 ppm. In some aspects, the preservative is present at a concentration from about 1 ppm to about 1000 ppm. In other aspects, the preservative is present at a concentration from about 10 ppm to about 300 ppm. In other embodiments, the preservative is present at a concentration from about 10 ppm to about 200 ppm. In certain aspects, the preservative is present at a concentration of less than about 0.001% (w/v). In other aspects, the preservative is present at a concentration of at least about 0.001% (w/v). In certain embodiments, the preservative is present at a concentration from about 0.001% (w/v) to about 1% (w/v). In certain aspects, the preservative is benzalkonium chloride. In some aspects, the benzalkonium chloride is present at a concentration from about 0.002% (w/v) to about 0.02% (w/v). In specific aspects, the benzalkonium chloride is present at a concentration of about 0.0075% (w/v).

The topical ophthalmic compositions of the present invention may be prepared by techniques known to those skilled in the art. The topical ophthalmic compositions of the invention may be an aqueous solution, emulsion or suspension or may be a dried preparation. In some aspects, the topical ophthalmic compositions of the invention may be desiccated or lyophilized, for example, by freeze-drying or spray drying for storage or formulations purposes. In certain aspects, a solid composition of the invention is subsequently reconstituted into liquid compositions by the addition of an appropriate liquid carrier prior to administering to a subject in need thereof.

The invention further relates to methods of treating an ocular condition in a subject in need of treatment thereof, comprising administering one or more topical ophthalmic compositions of the invention. As used herein, the term “ocular condition” may refer to any condition, disease, or impairment, which affects or involves the eye or one of the parts or regions of the eye, and includes optical issues causing refractive errors in the eye. Ocular conditions include, but are not limited to presbyopia, myopia, progressive myopia, pathologic myopia, amblyopia, cycloplegia, mydriasis, allergic conjunctivitis, conjunctival hyperemia, red eye, glaucoma, ocular hypertension, night vision symptoms post refractive surgery (e.g., glare, halos or starbursts around lights), accommodative esotropia, glaucoma, ocular hypertension, accommodative insufficiency, hyperopia, anisocoria, astigmatism, amblyopia, Adie's tonic pupil, or other causes of parasympathetic denervation, complications arising after refractive surgery, such as decentered ablations following LASIK or PRK, LASIK undercorrections, LASIK overcorrections, corneal scars, hazing, and refractive errors. In specific embodiments, the ocular condition is presbyopia.

The invention further provides methods of treating presbyopia in a subject in need of treatment thereof. The methods comprise administering to at least one eye of the subject a therapeutically effective amount of one or more topical ophthalmic compositions comprising one or more active components. The active components in the topical ophthalmic compositions include, but are not limited to carbachol, phospholine iodide, and pharmaceutically acceptable salts thereof. The topical ophthalmic compositions also include a buffer. In certain aspects, the topical ophthalmic compositions have a pH of about 3.0 to about 5.5. In other aspects, the topical ophthalmic compositions do not contain a viscosity-enhancing component.

“Presbyopia” is farsightedness caused by loss of elasticity of the lens of the eye, occurring typically in middle and old age. It is a condition associated with the aging of the eye that results in progressively worsening ability to focus clearly (particularly at close distance). Symptoms include difficulty reading small print, having to hold reading material farther away, headaches, and eyestrain. Most people begin to notice the effects of presbyopia sometime after age 40, when they start having trouble seeing small print clearly—including text messages on their phone. Application of cholinergic agonists (miotic agents) in these subjects is beneficial as the miosis resulting from sphincter muscle contraction creates a “pin-hole effect” that may potentially improve the near and intermediate vision by increasing the depth of field. These cholinergic agonists can thus be used for the treatment of presbyopia, although most effective dosing frequency and dose concentrations have not been defined.

As used herein, the term “treating” refers to both therapeutic measures and prophylactic or preventative measures, wherein the objective is to prevent, slow down (lessen), or ameliorate the progression of a disease (e.g., presbyopia). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total).

As used herein, the term “subject” refers to any individual, e.g., a mammal, for whom diagnosis, prognosis, or therapy is desired. The term “subject” may mean a human or non-human mammal affected, likely to be affected, or suspected to be affected with an ocular condition or disease. Although the topical ophthalmic compositions provided herein are principally directed to compositions which are suitable for administration to humans, the skilled artisan will understand that such compositions are generally suitable for administration to subjects of all sorts. In certain aspects, the subject is a mammal. In some aspects, a mammal includes, without limitation, primates, such as humans, monkeys and apes, and non-primates such as domestic animals, including laboratory animals, sports animals, farm animals, and household pets (e.g., cats, dogs, swine, cattle, cows, sheep, goats, horses, guinea pigs, rabbits, rats, mice), and non-domestic animals, such as wildlife, birds, or the like.

As used herein, the term “a subject in need thereof” includes subjects, such as mammalian subjects, that would benefit from administration of a topical ophthalmic composition of the invention. Subjects in need of treatment include, without limitation, those already with the condition or disorder as well as those prone to having the condition or disorder, or those in which the condition or disorder is to be prevented, ameliorated, or reversed.

By “therapeutically effective” is meant that the topical ophthalmic compositions are able to exert a statistically significant medically beneficial effect when used as prescribed or directed, as compared to a placebo.

The term “administer,” or “administering” as it applies to, for example, a subject in need of the topical ophthalmic compositions of the invention, refers to contact of, for example, the topical ophthalmic composition of the invention to at least one eye of the subject. In the context of a cell, administration includes contact (e.g., in vitro or ex vivo) of the topical ophthalmic compositions of the invention to the cell, as well as contact of the topical ophthalmic compositions of the invention with a fluid, where the fluid is in contact with the cell.

In certain embodiments, the topical ophthalmic compositions of the invention are administered to only one eye of a subject in need thereof. In other embodiments, the topical ophthalmic compositions of the invention are administered to at least one eye of a subject. In yet other embodiments, the topical ophthalmic compositions of the invention are administered to both eyes of a subject.

Normally a subject has a dominant eye and a non-dominant eye. The “dominant eye” is the eye that has a greater visual acuity and, therefore, dominates the depth vision. The “non-dominant eye” usually dominates the peripheral and spatial vision. Their interaction causes the brain to receive a three-dimensional image. Usually the dominant eye is the eye that is used to look through a microscope, a camera, or for any task in which only one eye is used. In certain embodiments, the topical ophthalmic compositions of the invention are administered to a non-dominant eye of a subject. In other embodiments, the topical ophthalmic compositions of the invention are administered to a dominant eye of a subject. In yet other embodiments, the topical ophthalmic compositions of the invention are administered to both the non-dominant eye and the dominant eye of the subject.

The topical ophthalmic compositions of the invention may be administered at several intervals in order to sustain therapeutic levels. For example, the topical ophthalmic compositions of the invention may be administered once daily, twice daily (BID), four times daily (QID) or more. In some embodiments, the topical ophthalmic compositions of the invention are administered once daily. In other embodiments, the topical ophthalmic compositions of the invention are administered twice daily.

In certain aspects, the topical ophthalmic compositions of the invention have a duration of action of at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 24 hours, as well as all intervening time points. In specific embodiments, the topical ophthalmic compositions of the invention have a duration of action greater than 10 hours, for example 12 hours, or even 24 hours. As used herein the term “duration of action” refers to the duration of time that an administered topical ophthalmic composition has an effect on at least one vision parameter (e.g., improvement of near vision), or ocular condition (e.g., presbyopia), or reduction of pupil diameter in a subject in need thereof. In certain embodiments, the topical ophthalmic compositions of the invention remain effective following administration for a period of time selected from the group consisting of at least about 6 hours, at least about 8 hours, at least about 10 hours, at least about 12 hours, and at least about 24 hours.

The topical ophthalmic compositions of the invention do not cause, or do not significantly cause vision blur when administered to a subject in need thereof. Additionally, when administered to subjects, the topical ophthalmic compositions of the invention do not cause, or do not significantly cause one or more one adverse effect that include, but are not limited to, ocular blurring, ocular discomfort, eye pain, brow ache, blurry vision, light sensitivity, ocular stinging, and ocular itching.

In certain embodiments, the topical ophthalmic compositions of the invention reduce incidence of at least one adverse effect compared to topical administration of a second ophthalmic composition comprising a viscosity-enhancing component and one or more active components selected from the group consisting of carbachol, phospholine iodide, and any pharmaceutically acceptable salt thereof. In certain cases, the adverse effects include, without limitation, ocular blurring, ocular discomfort, eye pain, brow ache, blurry vision, light sensitivity, ocular stinging, and ocular itching.

In certain aspects, the improved comfort associated with administering the topical ophthalmic compositions of the invention is due to the reduced viscosity of the compositions. In other aspects, the improved comfort associated with administering the topical ophthalmic compositions of the invention is due to the acidic pH of the compositions. In yet other aspects, the improved comfort associated with administering the topical ophthalmic compositions of the invention is due to both the reduced viscosity and the acidic pH of the compositions.

The invention additionally relates to methods for improvement of near vision in a subject with presbyopia in need of treatment thereof. The methods comprise administering to at least one eye of the subject a therapeutically effective amount of one or more topical ophthalmic compositions comprising one or more active components. The active components in the topical ophthalmic compositions include, but are not limited to carbachol, phospholine iodide, and pharmaceutically acceptable salts thereof. The topical ophthalmic compositions also include a buffer. In certain aspects, the topical ophthalmic compositions have a pH of about 3.0 to about 5.5. In other aspects, the topical ophthalmic compositions do not contain a viscosity-enhancing component.

The invention further provides methods of improving near reading speed in a subject with presbyopia in need of treatment thereof. The methods comprise administering to at least one eye of the subject a therapeutically effective amount of one or more topical ophthalmic compositions comprising one or more active components. The active components in the topical ophthalmic compositions include, but are not limited to carbachol, phospholine iodide, and pharmaceutically acceptable salts thereof. The topical ophthalmic compositions also include a buffer. In certain aspects, the topical ophthalmic compositions have a pH of about 3.0 to about 5.5. In other aspects, the topical ophthalmic compositions do not contain a viscosity-enhancing component.

The invention further relates to methods for reducing pupil diameter in a subject with presbyopia in need of treatment thereof. The methods comprise administering to at least one eye of the subject a therapeutically effective amount of one or more topical ophthalmic compositions of the invention.

The normal pupil size in adults varies from 2 to 4 mm in diameter in bright light to 4 to 8 mm in the dark. The pupils are generally equal in size. They constrict to direct illumination (direct response) and to illumination of the opposite eye (consensual response). The pupil dilates in the dark. Both pupils constrict when the eye is focused on a near object (accommodative response).

In certain embodiments, the methods of the invention result in a reduction of pupil diameter of at least about 10% of baseline pupil diameter over a time period of about 10 minutes to about 180 minutes following administration of the topical ophthalmic compositions of the invention. In other embodiments, the methods of the invention result in a reduction of pupil diameter of at least about 80% of baseline pupil diameter over a time period of about 10 minutes to about 180 minutes following administration of the topical ophthalmic compositions of the invention. In additional embodiments, the methods of the invention result in a reduction of pupil diameter of about 10% to about 90% of baseline pupil diameter over a time period of about 10 minutes to about 180 minutes following administration of the topical ophthalmic compositions of the invention. In specific embodiments, the methods of the invention result in a reduction of pupil diameter of about 20% to about 30% of baseline pupil diameter over a time period of about 30 minutes to about 120 minutes following administration of the topical ophthalmic compositions of the invention. In other embodiments, the methods of the invention result in a reduction of pupil diameter of about 10% of baseline pupil diameter at about 180 minutes following administration of the topical ophthalmic compositions of the invention.

The invention further provides methods of improving at least one vision parameter in a subject in need thereof, comprising administering to at least one eye of the subject one or more topical ophthalmic compositions of the invention. As used herein, the term “vision parameter” refers to any characteristic in a subject's vision that may be measured and is susceptible to being improved by the topical ophthalmic compositions and methods described herein. Vision parameters include, but are not limited to, near vision acuity, intermediate visual acuity, distance visual acuity, night vision, day vision, optical aberrations (e.g., glare, light scattering), and uncorrected refractive errors. Additional examples of vision parameters include, without limitation, night time glare, post-LASIK “star burst” glare, visual “halos” seen around light sources, and accommodative insufficiency.

The term “improving vision parameter,” including, but not limited to, near, intermediate, and/or distance visual acuity, may for example be reflected in the increase of number of letters correctly read at any time point post dosing, the increase in the average letter change, or 2-line or 3-line improvement, all from baseline (i.e., from pre-treatment). Night vision improvement may be reflected in visual improvement for subjects in dim or dark lighting (e.g., under mesopic or scotopic conditions). Day vision improvement may be reflected in visual improvement for subjects in bright lighting as found during daylight hours or in sunshine (e.g., under photopic conditions). Vision improvement using the methods described herein may also be achieved in combination with or when using other visual aids and devices (e.g., those used for treating presbyopia), including but not limited to reading glasses, lens modifying medications, and surgical presbyopic options including intraocular lenses (IOLs).

In certain embodiments, methods of treatment using the topical ophthalmic compositions described herein result in an at least 2-line improvement from baseline under the condition of photopic, high contrast uncorrected near visual acuity (UNVA). As used herein, the term “photopic” vision is the vision of the eye under well-lit conditions (luminance level 10 to 10⁸ cd/m²). In humans and other animals, photopic vision allows color perception, mediated by cone cells, and a significantly higher visual acuity and temporal resolution than available with scotopic vision (the vision of the eye under low-light conditions; luminance level 10⁻³ to 10⁻⁶ cd/m²).

As used herein, the term “uncorrected near visual acuity” (UNVA) refers to a subject's ability, without any vision aid (such as eyeglasses or contact lenses), to see the details of objects within arm's distance from the body (e.g., at 33-41 cm away from the eye).

In some embodiments, methods of treatment using the topical ophthalmic compositions described herein result in an at least 3-line improvement from baseline under the condition of photopic, high contrast UNVA. In other embodiments, methods described herein result in an increase in the average letter change from baseline under the condition of photopic, high contrast UNVA.

The term “improvement from baseline” refers to the increase from pre-treatment in the number of letters correctly read at certain post treatment time point. As used herein, the term “2-line improvement from baseline” or “3-line improvement from baseline” or similar improvement from baseline refers to a subject's ability to read 2 or 3 more lines of letters on a standard chart (e.g., Snellen, ETDRS, Logarithmic Visual Acuity Chart, etc.) after treatment with a topical ophthalmic composition of the invention when comparing to the number of lines readable before treatment.

In certain embodiments, methods of treatment using the topical ophthalmic compositions described herein result in an at least 2-line improvement from baseline under the condition of mesopic, high contrast UNVA. As used herein, the term “mesopic” vision refers to a combination of photopic vision and scotopic vision in low but not quite dark lighting situations. Mesopic light levels range from luminances of approximately 0.001 to 3 cd m⁻². Most night-time outdoor and traffic lighting scenarios are in the mesopic range. The human eye uses scotopic vision under low-light conditions and mesopic vision in intermediate conditions. Humans see differently at different light levels. This is because under high light levels typical during the day (photopic vision), the eye uses cones to process light. Under very low light levels, corresponding to moonless nights without electric lighting (scotopic vision), the eye uses rods to process light. At many night-time levels, a combination of both cones and rods supports vision. Photopic vision facilitates excellent color discrimination ability, whereas colors are indistinguishable under scotopic vision. Mesopic vision falls between these two extremes. In most night-time environments, there is enough ambient light at night to prevent true scotopic vision.

In some embodiments, methods of treatment using the topical ophthalmic compositions described herein result in an at least 3-line improvement from baseline under the condition of mesopic, high contrast UNVA. In other embodiments, methods described herein result in an increase in the average letter change from baseline under the condition of mesopic, high contrast UNVA.

In certain embodiments, methods of treatment using the topical ophthalmic compositions described herein result in an at least 2-line improvement from baseline under the condition of photopic, high contrast uncorrected distance visual acuity (UDVA). As used herein, the term “uncorrected distance visual acuity” (UDVA) refers to a subject's ability, without any vision aid (such as eyeglasses or contact lenses), to see the details of objects beyond arm's distance from the body (e.g., greater than 4 meters away from the eye).

In some embodiments, methods of treatment using the topical ophthalmic compositions described herein result in an at least 3-line improvement from baseline under the condition of photopic, high contrast UDVA. In other embodiments, methods described herein result in an increase in the average letter change from baseline under the condition of photopic, high contrast UDVA.

In certain embodiments, methods of treatment using the topical ophthalmic compositions described herein result in an at least 2-line improvement from baseline under the condition of mesopic, high contrast distance-corrected near visual acuity (DCNVA). As used herein, the term “distance corrected near visual acuity” (DCNVA) refers to a subject's ability to see the details of objects within arm's distance from the body (e.g., at 33-41 cm away from the eye), with the use of vision aids such as eyeglasses or contact lenses that correct for distance vision issues.

In some embodiments, methods of treatment using the topical ophthalmic compositions described herein result in an at least 3-line improvement from baseline under the condition of mesopic, high contrast DCNVA. In other embodiments, methods described herein result in an increase in the average letter change from baseline under the condition of mesopic, high contrast DCNVA. In yet other embodiments, methods described herein result in an at least 3-line improvement from baseline under the condition of photopic, high contrast DCNVA. In additional embodiments, methods described herein result in an at least 2-line improvement from baseline under the condition of photopic, high contrast DCNVA. In further embodiments, methods described herein result in an increase in the average letter change from baseline under the condition of photopic, high contrast DCNVA.

In certain embodiments, methods of treatment using the topical ophthalmic compositions described herein result in an at least 2-line improvement from baseline under the condition of mesopic, high contrast distance-corrected intermediate visual acuity (DCIVA). As used herein, the term “distance-corrected intermediate visual acuity” (DCIVA) may be used to refer to a subject's ability to see the details of objects at intermediate distances with the use of vision aids such as eyeglasses or contact lenses that correct for distance vision issues.

In some embodiments, methods of treatment using the topical ophthalmic compositions described herein result in an at least 3-line improvement from baseline under the condition of mesopic, high contrast DCIVA. In other embodiments, methods described herein result in an increase in the average letter change from baseline under the condition of mesopic, high contrast DCIVA. In yet other embodiments, methods described herein result in an at least 2-line improvement from baseline under the condition of photopic, high contrast DCIVA. In additional embodiments, methods described herein result in an at least 3-line improvement from baseline under the condition of photopic, high contrast DCIVA. In further embodiments, methods described herein result in an increase in the average letter change from baseline under the condition of photopic, high contrast DCIVA.

### EXAMPLES

### Example 1: Topical Ophthalmic Compositions of the Invention

The following Tables provide examples of the topical ophthalmic compositions of the invention.

TABLE 1
Compositions of Carbachol

| | Multi-dose with | | | |
|---|---|---|---|---|
| | preservative | | Preservative free | |
| Formulation | Target | | Target | |
| Type | Conc- | Expected | Conc- | Expected |
| Ingredient | entration | Range | entration | Range |
| (% w/v)¹ | (% w/w) | (% w/w) | (% w/w) | (% w/w) |
| Carbachol | 0.6 | 0.1 to 1.0 | 0.6 | 0.1 to 1.0 |
| Benzalkonium | 0.0075 | 0.002 to 0.02 | 0 | 0 |
| chloride | | | | |
| Boric acid | 1.0 | 0.1 to 1.5 | 1.0 | 0.1 to 1.5 |
| Sodium citrate | 0.015 | 0.01 to 0.1 | 0.015 | 0.01 to 0.1 |
| dihydrate | | | | |
| Sodium | 0.37 | 0.1 to 0.9 | 0.37 | 0.1 to 0.9 |
| chloride | | | | |
| Hydrochloric | pH 3.0-5.5 | pH 3.0-5.5 | pH 3.0-5.5 | pH 3.0-5.5 |
| acid and/or | | | | |
| Sodium | | | | |
| hydroxide | | | | |
| Purified water | QS | QS | QS | QS |

¹The density of formulations are within 0.99-1.00 g/mL at 25° C. Hence, the composition ingredients in % w/v is equivalent to the % w/w.

TABLE 2
Additional Compositions of Carbachol

| | Multi-dose with | | | |
|---|---|---|---|---|
| | preservative | | Preservative free | |
| Formulation | Target | | Target | |
| Type | Conc- | Expected | Conc- | Expected |
| Ingredient | entration | Range | entration | Range |
| (% w/v)¹ | (% w/w) | (% w/w) | (% w/w) | (% w/w) |
| Carbachol | 3.0 | 2.5 to 3.5 | 3.0 | 2.5 to 3.5 |
| Benzalkonium | 0.0075 | 0.002 to 0.02 | 0 | 0 |
| chloride | | | | |
| Boric acid | 1.0 | 0.1 to 1.5 | 1.0 | 0.1 to 1.5 |
| Sodium citrate | 0.015 | 0.01 to 0.1 | 0.015 | 0.01 to 0.1 |
| dihydrate | | | | |
| Sodium | 0.37 | 0.1 to 0.9 | 0.37 | 0.1 to 0.9 |
| chloride | | | | |
| Hydrochloric | pH 3.0-5.5 | pH 3.0-5.5 | pH 3.0-5.5 | pH 3.0-5.5 |
| acid and/or | | | | |
| Sodium | | | | |
| hydroxide | | | | |
| Purified water | QS | QS | QS | QS |

¹The density of formulations are within 0.99-1.00 g/mL at 25° C. Hence, the composition ingredients in % w/v is equivalent to the % w/w.

TABLE 3
Compositions of Phospholine iodide

| | Multi-dose | | | |
|---|---|---|---|---|
| | with preservative | | Preservative free | |
| Formulation | Target | | Target | |
| Type | Conc- | Expected | Conc- | Expected |
| Ingredient | entration | Range | entration | Range |
| (% w/v)¹ | (% w/w) | (% w/w) | (% w/w) | (% w/w) |
| Phospholine | 0.06 | 0.01 to 0.25 | 0.06 | 0.01 to 0.25 |
| iodide | | | | |
| Benzalkonium | 0.0075 | 0.002 to 0.02 | 0 | 0 |
| chloride | | | | |
| Boric acid | 1.0 | 0.1 to 1.5 | 1.0 | 0.1 to 1.5 |
| Sodium | 0.015 | 0.01 to 0.1 | 0.015 | 0.01 to 0.1 |
| citrate | | | | |
| dihydrate | | | | |
| Sodium | 0.37 | 0.1 to 0.9 | 0.37 | 0.1 to 0.9 |
| chloride | | | | |
| Hydrochloric | pH 3.0-5.5 | pH 3.0-5.5 | pH 3.0-5.5 | pH 3.0-5.5 |
| acid and/or | | | | |
| Sodium | | | | |
| hydroxide | | | | |
| Purified water | QS | QS | QS | QS |

¹The density of formulations are within 0.99-1.00 g/mL at 25° C. Hence, the composition ingredients in % w/v is equivalent to the % w/w.

### Example 2—Effect of Pilocarpine on Pupil Diameter in Rabbit Animal Model

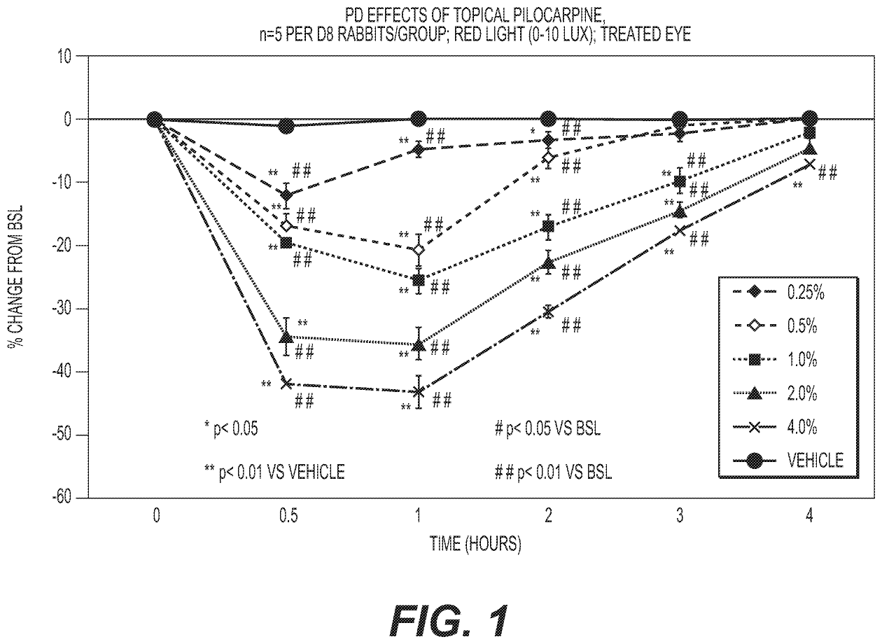

Dose solutions with concentration range of 0.25% to 4% pilocarpine were prepared and dosed in rabbits. The effect on pupil diameter was measured and recorded as the change from baseline. A dose of 1.25% w/w resulted in a 20-30% reduction from baseline pupil diameter over 0.5 to 2 hours, with an approximately 10% reduction from baseline up to 3 hours (FIG. 1).

### Example 3—Effect of Carbachol on Pupil Diameter in Rabbit Animal Model

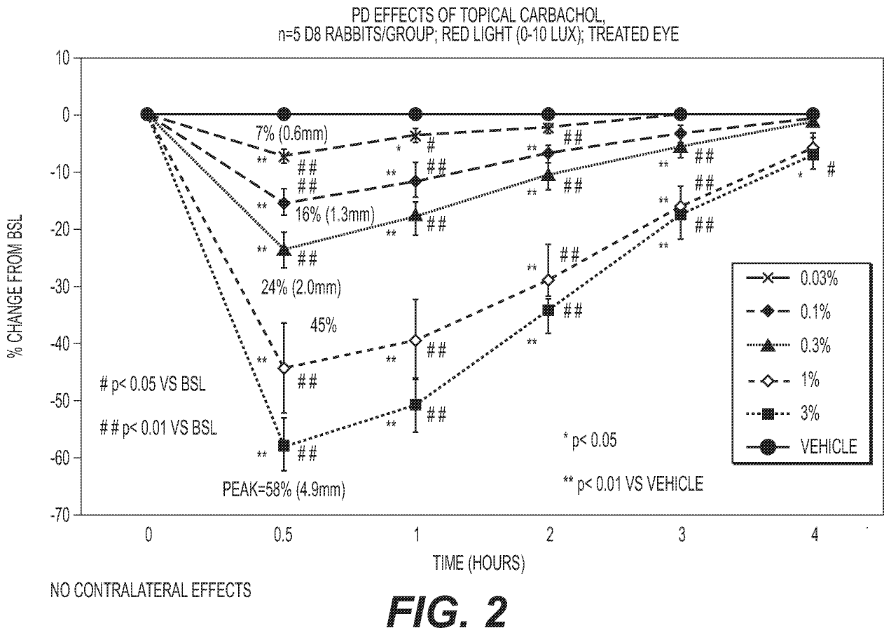

Dose solutions with concentration range of 0.03% to 3% carbachol were prepared and dosed in rabbits. The effect on pupil diameter was measured and recorded as the change from baseline. The results show that the dose range of carbachol that would most closely match the optimum dose response measured for pilocarpine in Example 2 (approximately 20-30% reduction from baseline pupil diameter over 0.5 to 2 hours with, an approximately 10% reduction from baseline up to 3 hours) corresponds to approximately 0.6% w/w. The expected efficacious range of carbachol resulting in the aforementioned pupil size reduction would correspond to approximately 0.1 to 1% w/w carbachol (FIG. 2).

### Example 4—Effective Dose of Phospholine Iodide

The effective dose of phospholine iodide that would most closely match the optimum dose response measured for pilocarpine in Example 2 (approximately 20-30% reduction from baseline pupil diameter over 0.5 to 2 hours, with an approximately 10% reduction from baseline up to 3 hours) corresponds to approximately 0.06% w/w. The expected efficacious range of phospholine iodide resulting in the aforementioned pupil size reduction would therefore correspond to approximately 0.01 to 0.25% w/w phospholine iodide.

While certain embodiments of the invention have been described, other embodiments may exist. While the specification includes a detailed description, the invention's scope is indicated by the following claims. Furthermore, while the specification has been described in language specific to structural features and/or methodological acts, the claims are not limited to the features or acts described above. Rather, the specific features and acts described above are disclosed as illustrative aspects and embodiments of the invention. Various other aspects, embodiments, modifications, and equivalents thereof which, after reading the description herein, may suggest themselves to one of ordinary skill in the art without departing from the spirit of the present invention or the scope of the claimed subject matter.

1. A topical ophthalmic composition comprising one or more active components selected from the group consisting of carbachol, phospholine iodide, and pharmaceutically acceptable salts thereof, and a buffer, wherein the composition has a pH of about 3.0 to about 5.5 and does not contain a viscosity-enhancing component.

2. A topical ophthalmic composition comprising one or more active components selected from the group consisting of carbachol, phospholine iodide, and pharmaceutically acceptable salts thereof, and a buffer, wherein the composition has a pH of about 3.0 to about 5.5 and a viscosity from about 1 centipoise (cps) to about 10 cps.

3. The topical ophthalmic composition of claim 1, wherein the carbachol or phospholine iodide is present at a concentration from about 0.01% (w/v) to about 20% (w/v).

4. The topical ophthalmic composition of claim 3, wherein the carbachol or phospholine iodide is present at a concentration from about 0.01% (w/v) to about 10% (w/v).

5. The topical ophthalmic composition of claim 4, wherein the carbachol is present at a concentration from about 0.03% (w/v) to about 3.5% (w/v).

6. The topical ophthalmic composition of claim 5, wherein the carbachol is present at a concentration from about 0.1% (w/v) to about 1% (w/v).

7. The topical ophthalmic composition of claim 6, wherein the carbachol is present at a concentration of 0.6% (w/v).

8. The topical ophthalmic composition of claim 4, wherein the phospholine iodide is present at a concentration from about 0.01% (w/v) to about 0.25% (w/v).

9. The topical ophthalmic composition of claim 8, wherein the phospholine iodide is present at a concentration of 0.06% (w/v).

10. The topical ophthalmic composition of claim 1, wherein the buffer is selected from the group consisting of sodium citrate dehydrate buffer, phosphate buffer, borate buffer, borate citrate buffer, and lactate buffer.

11. The topical ophthalmic composition of claim 1, further comprising one or more osmolality agents.

12. The topical ophthalmic composition of claim 11, wherein the one or more osmolality agents is selected from the group consisting of glycerin, propylene glycol, mannitol, sorbitol, sodium chloride, potassium chloride and dextrose.

13. The topical ophthalmic composition of claim 1, further comprising a preservative.

14. The topical ophthalmic composition of claim 13, wherein the preservative is selected from the group consisting of benzalkonium chloride and a stabilized oxychloro complex.

15. The topical ophthalmic composition of claim 1, wherein the composition comprises carbachol as the sole active component.

16. The topical ophthalmic composition of claim 1, wherein the composition comprises phospholine iodide as the sole active component.

17. The topical ophthalmic composition of claim 1, wherein the topical ophthalmic composition remains effective following administration for a period of time selected from the group consisting of at least about 6 hours, at least about 8 hours, at least about 10 hours, at least about 12 hours, and at least about 24 hours.

18. The topical ophthalmic composition of claim 1, wherein the composition is administered once daily.

19. The topical ophthalmic composition of claim 1, wherein the composition is administered twice daily.

20. The topical ophthalmic composition of claim 1, wherein the composition is administered to both eyes of a subject.

21. The topical ophthalmic composition of claim 1, wherein the composition is administered to a nondominant eye of a subject.

22. The topical ophthalmic composition of claim 1, wherein the composition is administered to a dominant eye of a subject.

23. A method of treating presbyopia in a subject in need of treatment thereof, comprising administering to at least one eye of the subject a therapeutically effective amount of a topical ophthalmic composition comprising one or more active components selected from the group consisting of carbachol, phospholine iodide, and pharmaceutically acceptable salts thereof.

24. A method for improvement of near vision in a subject with presbyopia in need thereof, comprising administering to at least one eye of the subject a therapeutically effective amount of a topical ophthalmic composition comprising one or more active components selected from the group consisting of carbachol, phospholine iodide, and pharmaceutically acceptable salts thereof.

25. A method for reducing pupil diameter in a subject with presbyopia in need thereof, comprising administering to at least one eye of the subject a therapeutically effective amount of a topical ophthalmic composition comprising one or more active components selected from the group consisting of carbachol, phospholine iodide, and pharmaceutically acceptable salts thereof.

26. The method of claim 25, wherein the method results in a reduction of pupil diameter of about 20% to about 30% of baseline pupil diameter over a time period of about 30 minutes to about 120 minutes following administration of the topical ophthalmic composition.

27. The method of claim 25, wherein the method results in a reduction of pupil diameter of about 10% of baseline pupil diameter at about 180 minutes following administration of the topical ophthalmic composition.

28. The method of claim 23, wherein the topical ophthalmic composition comprises carbachol as the sole active component.

29. The method of claim 28, wherein the carbachol is present at a concentration from about 0.01% (w/v) to about 10% (w/v).

30. The method of claim 29, wherein the carbachol is present at a concentration from about 0.03% (w/v) to about 3.5% (w/v).

31. The method of claim 30, wherein the carbachol is present at a concentration from about 0.1% (w/v) to about 1% (w/v).

32. The method of claim 31, wherein the carbachol is present at a concentration of 0.6% (w/v).

33. The method of claim 23, wherein the topical ophthalmic composition comprises phospholine iodide as the sole active component.

34. The method of claim 33, wherein the phospholine iodide is present at a concentration from about 0.01% (w/v) to about 10% (w/v).

35. The method of claim 34, wherein the phospholine iodide is present at a concentration from about 0.01% (w/v) to about 0.25% (w/v).

36. The method of claim 35, wherein the phospholine iodide is present at a concentration of 0.06% (w/v).

37. The method of claim 23, wherein the topical ophthalmic composition has a pH of about 3.0 to about 5.5.

38. The method of claim 23, wherein the topical ophthalmic composition does not contain a viscosity-enhancing component.

39.-41. (canceled)
